(19) United States
(12) Patent Application Publication
(10) Pub. No.: US 2009/0124007 A1
(43) Pub. Date: May 14, 2009

(54) Method for the Simultaneous Primary-Isolation and Expansion of Endothelial Stem/Progenitor Cell and Mesenchymal Stem Cell Derived From Mammal Including Human Umbilical Cord

(75) Inventor: Jae Jin Cho, Seoul (KR)

Correspondence Address: GREENLEE WINNER AND SULLIVAN P C, 4875 PEARL EAST CIRCLE, SUITE 200, BOULDER, CO, 80301, US

(73) Assignee: Seoul National University Industry Foundation, Seoul (KR)

(21) Appl. No.: 12/085,053

(22) PCT Filed: Sep. 27, 2007
(86) PCT No.: PCT/KR2007/004698

§ 371 Date: May 14, 2008

(30) Foreign Application Priority Data
Nov. 15, 2006 (KR) 10-2006-0112609
Jan. 19, 2007 (KR) 10-2007-0006119

(51) Int. Cl. C12N 5/02

(52) U.S. Cl. 435/366; 435/378; 435/374; 435/383

(57) ABSTRACT

Disclosed herein is a method for isolating and culturing the endothelial stem/progenitor cells and mesenchymal stem cells derived from the umbilical cord of mammals, including human beings. More specifically, disclosed are a method for isolating and culturing endothelial stem/progenitor cells and mesenchymal stem cells at high purity from the umbilical cord of mammals, including human beings, and preferably from the human umbilical cord, as well as endothelial stem/progenitor cells and mesenchymal stem cells, isolated and cultured according to said method, and a method for freezing and thawing the isolated and cultured cells. According to the disclosed method, endothelial stem/progenitor cells and mesenchymal stem cells can be easily isolated and purified with high purity from umbilical cord, and can be cultured with high viability for a long period of time.

### TECHNICAL FIELD

The present invention relates to a method for isolating and culturing the endothelial stem/progenitor cells and mesenchymal stem cells derived from the umbilical cord of mammals, including human beings. More particularly, the present invention relates to a method for simultaneously isolating, identifying and culturing endothelial stem/progenitor cells and mesenchymal stem cells at high purity from the umbilical cord of mammals, including human beings, and preferably from the human umbilical cord.

### BACKGROUND ART

Stem cells have the capability to proliferate indefinitely and differentiate into all cells. Embryonic stem cells are most suitable for the definition of such stem cells, but adult stem cells have been studied as alternatives due to ethical problems.

Among adult stem cells, blood-derived stem cells, such as those derived from bone marrow and cord blood, have been most well studied. Blood-derived stem cells include hematopoietic stem cells and mesenchymal stem cells. Mesenchymal stem cells include very diverse cells compared to hematopoietic stem cells, and studies on the characteristics thereof are still insufficient. Furthermore, blood-derived mesenchymal stem cells are limited in the number of obtainable cells and have risks, such as a change in characteristics, when they are cultured.

Recent study results show the possibility that at least two kinds of stem cells, in addition to cord blood, may exist in human umbilical cord.

It is known that vascular endothelial progenitor cells are CD34+CD133+VEGF receptor-2+ cells and can be isolated from peripheral blood or cord blood (Science, Feb. 14, 1997; 275 (5302): 964-7; Blood. Feb. 1, 2000; 95(3): 952-8; 3 Clin Invest. February 2002; 109(3): 337-46). However, there are many disputes on the definition of such endothelial progenitor cells or on methods for culturing the cells (Blood. Mar. 1, 2007; 109(5); 1801-9. Epub Oct. 19, 2006). Recently, it was reported that endothelial progenitor cells exist in such endothelial cells (Blood. Apr. 1, 2005; 105(7): 2783-6. Epub Dec. 7, 2004). Clonal analysis demonstrated that the endothelial progenitor cells from endothelial cells have characteristics similar to those of endothelial progenitor cells obtained from peripheral blood or cord blood.

Meanwhile, there are reports indicating that mesenchymal stem cells were isolated from human umbilical cord. It was reported that mesenchymal stem cells can be isolated from the Wharton's jelly, vein or artery of human umbilical cord, and the marker of the mesenchymal stem cells can be expressed such that the mesenchymal stem cells can differentiate into osteoblasts, chondrocytes or adipocytes (Stem Cells. 2004; 22(7): 1330-7; Stem Cells. February 2005; 23(2): 220-9). Recently, it was also reported that mesenchymal stem cells can differentiate into nerve cells (Stem Cells. January 2006; 24(1): 115-24, Epub Aug. 11, 2005; Stem Cells. March 2006; 24(3): 781-92. Epub Oct. 13, 2005).

In studies conducted by the present invention, a method for simultaneously isolating and culturing endothelial progenitor cells and mesenchymal stem cells from human umbilical cord was established. Also, mesenchymal stem cells were isolated from tissue remaining after endothelial progenitor cells were simply isolated from the vein of the human umbilical cord.

### DISCLOSURE

### [Technical Problem]

Accordingly, the present inventors have conducted studies on a method for efficiently isolating and culturing endothelial stem/progenitor cells and mesenchymal stem cells and, as a result, have obtained highly pure endothelial stem/progenitor cells and mesenchymal stem cells by efficiently isolating endothelial stem/progenitor cells and mesenchymal stem cells from the umbilical cord of mammals, including human beings, and culturing the isolated cells in suitable culture conditions, thereby completing the present invention.

Therefore, it is an object of the present invention to provide a method for isolating and culturing endothelial stem/progenitor cells and mesenchymal stem cells at high purity from the umbilical cord of mammals, including human beings.

### [Technical Solution]

To achieve the above object, the present invention provides a method for isolating and culturing endothelial stem/progenitor cells and mesenchymal stem cells at high purity from the umbilical cord of mammals, including human beings.

Hereinafter, the present invention will be described in detail.

As used herein, the term “progenitor cell” refer to a cell committed to differentiate into a specific type of cell or to form a specific type of tissue, and the term “endothelial progenitor cell” means a cell which can secrete an angiogenic growth factor to contribute indirectly to blood vessel regeneration or can differentiate directly into a mature endothelial cell.

As used herein, the term “stem cell” refers to a master cell that can differentiate to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. As used herein, the term “stem cell” refers to a master cell that can differentiate to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. As used herein, the term “endothelial stem/progenitor cell” refers to a middle stage between an endothelial stem cell and a progenitor cell or a cell group consisting of a mixture of these cells.

As used herein, the term “mesenchymal stem cell” refers to vascular mural cells surrounding the basement membrane of the fine blood vessels and refers to cells which can be formed from smooth muscle cells, fibroblasts, endothelial cells or bone marrow or differentiate into smooth muscle cells, fibroblasts, osteoblasts, chondrocytes or adipocytes.

Preferably, the inventive method for isolating and culturing endothelial stem/progenitor cells and mesenchymal stem cells from umbilical cord comprises the steps of: (a) culturing any one selected from the umbilical cord-derived vascular endothelium, blood vessel and blood vessel-removed umbilical cord of mammals, including human beings, together with protease or protease and DNA-degrading enzyme; (b) scratching the cultured endothelium of step (a) with a scraper to collect a cell mass, and collecting a supernatant of the cultured blood vessel and blood vessel-removed umbilical core of step (a); (c) isolating and purifying endothelial stem/progenitor cells from the cell mass of step (b), and isolating and purifying mesenchymal stem cells from the supernatant of step (b); and (d) culturing the endothelial stem/progenitor cells or mesenchymal stem cells, isolated and purified in the step (c).

The mammals in the step (a) may include human beings, monkeys, pigs, horses, cows, sheep, dogs, cats, mice and rats, but are preferably human beings. The umbilical cord of mammals, including human beings, is collected immediately after delivery, and can be transported at room temperature after it is immersed in a HBSS (Hank's balanced salt solution, JBI, 003-02) solution containing a high concentration of antibiotics in order to prevent contamination during transportation and storage.

The umbilical cord-derived vascular endothelium of mammals, including human beings, can be obtained by longitudinally cutting the vein of the umbilical cord. As used herein, the phrase “umbilical cord-derived blood vessel of mammals, including human beings” refers to an artery separated by cutting along the Wharton's jelly of the umbilical cord, and the term “blood vessel-removed umbilical cord” refers to an umbilical cord remaining after the artery is separated.

The protease is preferably collagenase and/or pronase. The collagenase is an enzyme degrading extracellular matrix protein collagen.

The DNA-degrading enzyme is preferably DNase.

I. Isolation and Culture of Umbilical Cord-Derived Endothelial Stem/Progenitor Cells

The culture of vascular endothelium in the step (a) can be carried out by treating vascular endothelium with collagenase and culturing the collagenase-treated endothelium at 35-38° C. for 15-20 minutes, and preferably at 37° C. for 20 minutes.

In the step (b), an endothelial cell mass can be obtained in an easy and efficient manner by simply scratching the collagenase-treated endothelium. Particularly, for the high-purity isolation of endothelium stem/progenitor cells, it is required to use a suitable force to scratch endothelium with a scraper.

In the step (c), endothelial stem/progenitor cells are isolated and purified from the endothelial cell mass, obtained in the step (b). The isolation and purification of endothelial stem/progenitor cells in the step (c) can be carried out by culturing the cell mass of step (b) in a water bath, filtering and washing the cultured cells, suspending the washed cells in a culture medium, and seeding the cell suspension in a culture dish.

The water-bath culture of the cell mass can be carried out with shaking at 35-38° C. for 20-40 minutes, and preferably 37° C. for about 30 minutes. Also, during the water-bath culture, a step of weakly shaking the culture medium at an interval of 4-6 minutes may additionally be included.

The filtration of the cultured cells can be performed by passing the cells through a mesh having a pore size of 70-100 μm, and preferably about 70 μm, to remove impurities. For this reason, the endothelial stem/progenitor cells of the present invention can be prevented from being contaminated with other cells, such as fibroblasts, and can be isolated and purified with a purity of more than 90%.

The washing of the cultured cells can be carried out by treating the cultured cells with a medium and centrifuging the cell-containing medium to remove the supernatant.

The step (d) is a step of culturing the endothelial stem/progenitor cells, isolated and purified in the step (c). In the step (d), the isolated and purified endothelial stem/progenitor cells can be can be cultured by suspending the cells in a medium and seeding the suspended cells in a culture dish.

The culture medium means a medium capable of supporting the ex vivo growth and survival of the endothelial stem/progenitor cells, and examples thereof include all conventional media, which are suitable for the culture of endothelial stem/progenitor cells and are used in the art. The medium for use in the cell culture preferably contains a carbon source, a nitrogen source and trace elements. As this medium, a medium containing RPMI 1640, FBS, insulin, hydrocortisone, heparin and an endothelial cell growth factor is preferably used. Most preferably, a medium shown in Table 1 below may be used.

The inventive method may additionally comprise a step of subculturing the cells, when the cells reach a confluency of 60-80%, and preferably 70%, after the initiation of the culture in the step (d).

The subculture step comprises the steps of: (i) washing the isolated and purified cells; (ii) placing the cells of step (i) in a culture dish, adding trypsin/EDTA to the cells, and then incubating the cells in a CO₂ incubator; (iii) applying a light impact to the culture dish of step (ii) to physically detach the cells from the dish, adding trypsin/EDTA and the same amount of medium to the cells to inactivate the cells, and then collecting the cells; and (iv) centrifuging the cells at low temperature to remove the supernatant, re-suspending the centrifuged cells in a culture medium, and transferring the cell-containing medium to another culture dish.

The washing in the step (i) can be performed by completely removing the remaining FBS using RPMI 1640.

The addition of trypsin/EDTA in the step (i) is preferably carried by adding trypsin/EDTA warmed in a water bath at 35-38° C. for 5-15 minutes.

Also, the incubation in the step (ii) can be carried out for 0.5-1.5 minutes.

The centrifugation in the step (iv) can be carried out at a temperature of 3-5° C., and preferably 4° C. The subculture step may additionally comprise, after the centrifugation in the step (iv), a step of staining the cells from which the supernatant has been removed, and monitoring the number or viability of the cells.

The endothelial stem/progenitor cells obtained according to the above-described method of the present invention are characterized in that they do not express Desmin and α-SMA (α-smooth muscle actin), highly express CD31, CD34 and vWF (von Willebrand Factor), and have the ability to absorb LDL (low density lipoprotein).

II. Isolation and Culture of Umbilical Cord-Derived Mesenchymal Cells

The method for isolating and culturing umbilical cord-derived mesenchymal cells according to the present invention is characterized in that human umbilical cord-derived mesenchymal cells can be obtained in an easy and efficient manner by adding several enzymes to blood vessels separated from umbilical cord, and to umbilical cord from which blood vessels have been separated.

More specifically, in the step (a), the culture of the blood vessel and the blood vessel-removed umbilical cord can be performed by treating said blood vessel or umbilical cord with collagenase, pronase or DNase and culturing the treated blood vessel or umbilical cord at 35-38° C. for 2-6 hours, and preferably at 37° C. for 4 hours.

In the step (b), the supernatant produced after completion of the culture of step (a) is collected.

In the step (c), the supernatant, obtained in the step (b), is filtered and washed to collect the cultured cells. The filtration can be performed by adding less than 50 ml of HBSS to order to efficiently collect cell mass from the supernatant obtained in the step (b), centrifuging the resulting supernatant to collect mesenchymal stem cells, and passing the mesenchymal stem cells through a mesh having a pore size of 10-100 μm, and preferably about 70 μm, to remove impurities. This filtration process according to the present invention can prevent the mesenchymal stem cells from being contaminated with other cells such as fibroblasts and reduce competition with other cells, thus increasing the viability of the mesenchymal stem cells in a culture dish. Particularly, the use of this method allows human umbilical cord-derived mesenchymal stem cells to be isolated and purified with a purity of more than 90%.

In the step (d), the mesenchymal stem cells, isolated and purified in the step (c), are suspended in a culture medium and seeded and cultured in a culture dish. Examples of the culture medium include all media which support the growth and survival of mesenchymal stem cells ex vivo. Preferably, a medium containing DMEM and FBS at a volume ratio of 4:1 may be used in the present invention.

The inventive method may additionally comprise a step of subculturing the cells, when the cells reach a confluency of 60-80%, and preferably 70%, after the initiation of the culture in the step (d).

The subculture can be performed in the steps (i) to (iv) described for the endothelial stem/progenitor cells. The washing of the cells in the step (i) can be carried out by removing the medium and then washing the cells with DMEM to completely remove the remaining FBS. The step (ii) is preferably carried out by adding 0.05% trypsin/EDTA, warmed in a water bath at 35-38° C. for about 5-15 minutes, and then incubating the cells in a CO₂ incubator for 0.5-1.5 minutes. Also, the centrifugation of the cells in the step (iv) can be carried out at a temperature of 3-5° C., and preferably 4° C. Moreover, the subculture process may additionally comprise, after the centrifugation, a step of staining the cells from which the supernatant has been removed, and monitoring the number or viability of the cells.

The umbilical cord-derived mesenchymal stem cells obtained according to the above-described method of the present invention are characterized in that the expression of CD29, CD44, CD73, CD90, CD105, α-SMA (α-smooth muscle actin) and NG2 (NG2 Chondroitin Sulfate Proteoglycan) is outstanding.

III. Freezing and Thawing of Umbilical Cord-Derived Endothelial Stem/Progenitor Cells and Mesenchymal Stem Cells

The umbilical cord-derived endothelial stem/progenitor cells and mesenchymal stem cells, isolated and cultured according to the method of the present invention, can be frozen and stored according to a method comprising steps of:
(a) suspending the umbilical cord-derived endothelial stem/progenitor cells and mesenchymal stem cells in a freezing medium, placing the cell suspension in a freezing vial, and placing the cell-containing vial in a cryogenic box, stored at room temperature; and
(b) storing the cell-containing vial in the cryogenic box at a temperature ranging from −75 to −85° C. for 20-30 hours, and then transferring and storing the cell-containing vial in a liquid nitrogen (LN2) tank.

In the step (a), about 1-2×10⁶ cells are prepared, and the freezing medium contains DMSO, FBS and RPMI1640 at a ratio of 5:10:35, but the scope of the present invention is not limited thereto.

On the other hand, the umbilical cord-derived endothelial stem/progenitor cells and mesenchymal stem cells, frozen according to the above-described method of the present invention, can be thawed according to a method comprising the steps of:
(a) rapidly thawing the freezing vial at a temperature of 35-38° C., and transferring the freezing vial to a clean bench before it is completely thawed;
(b) adding the cells, obtained in the step (a), drop-by-drop to a thawing medium using a pipette, and then centrifuging the cell-containing medium at a temperature of 2-5° C.; and
(c) suspending the cells in a culture medium and seeding the suspended cells in a culture dish. Herein, the thawing medium preferably contains FBS and DMEM at a volume ratio of 2:8, but the scope of the present invention is not limited thereto.

### DESCRIPTION OF DRAWINGS

FIG. 1 shows photographs of endothelial stem/progenitor cells and a growth curve of the cells. In FIG. 1, (A), (B) and (C) indicate 200× photographs of the cells at passage 0, passage 5 and passage 11, respectively. Photographs included in the 200× photographs are 400× photographs. The cells show the typical cobble-stone appearance of endothelial cells. (D) shows the number of cells, which can be obtained when 10,000 cells are cultured to passage 11.

FIG. 2 shows the results of FACS analysis conducted to examine the expression of CD31 and CD34, which are the markers of the inventive endothelial stem/progenitor cells, in which the FACS analysis was conducted during early culture. It can be seen that the expression of CD31 is maintained, whereas the expression of CD34 is rapidly reduced with the progression of passages.

FIG. 3 shows the results of analysis for the expression of CD31 and vWF, which are the inventive endothelial stem/progenitor cells, in which the analysis was conducted during early culture. In FIG. 5, A to C show the expression of CD31 at passages 0-3, and D to F show the expression of vWF at passages 0-3. G to L show the results of confocal microscopic observation at passage 7, and it can be seen that CD31 appears on the cell surface, and vWF is observed in the cytoplasm. The nuclei were stained with DAPI (blue).

FIG. 4 shows the results of analysis conducted to examine the LDL absorption ability and ex vivo angiogenic activity of the endothelial stem/progenitor cells of the present invention. In FIG. 4, (B) is a 200× fluorescent microphotograph of cells treated with Dil-Ac-LDL at 37° C. for 4 hours. (A) shows the results of FACS analysis of cells stained with CD31. It can be seen that 97% of cells absorbed LDL and, at the same time, expressed CD31. C to E are photographs showing the results of observation of 10,000 cells cultured on Matrigel in a 96-well plate. It can be seen that cells were linked with each other to form a vascular appearance.

FIG. 5 shows photographs of human umbilical cord-derived mesenchymal stem cells and a growth curve of the cells. In FIG. 5, (A), (B) and (C) indicate 200× photographs of the cells at passage 0, passage 5 and passage 11, respectively. It can be seen that the cells have an appearance similar to the typical cobble-stone appearance of endothelial cells. (D) shows the number of cells, which can be obtained when 10,000 cells are cultured to passage 11.

FIG. 6 shows the results of FACS analysis of the inventive human umbilical cord-derived mesenchymal stem cells, in which the FACS analysis was conduced during the cell culture. It can be seen that most of the cells expressed CD29, CD44, CD73, CD90 and CD105, which are the markers of mesenchymal stem cells. However, it can be seen that the mesenchymal stem cells did not substantially express CD31, which is the marker of endothelial cells, or CD34, CD45 and CD117, which are the markers of hematopoietic cells.

FIG. 7 shows the results of immunohistochemical staining of the inventive human umbilical cord-derived mesenchymal stem cells. It can be seen that the human umbilical cord-derived mesenchymal stem cells of the present invention expressed α-SMA (A-D) and NG-2 (I-L) at passages 2, 4, 8 and 10, but did not express desmin (E-H).

FIG. 8 shows the results of immunohistochemical staining of the inventive human umbilical cord-derived mesenchymal stem cells. As shown in FIG. 8, when the human umbilical cord-derived mesenchymal stem cells were stained simultaneously with α-SMA and NG-2, α-SMA could be observed in the cytoplasm, and NG-2 could be observed on the cell surface.

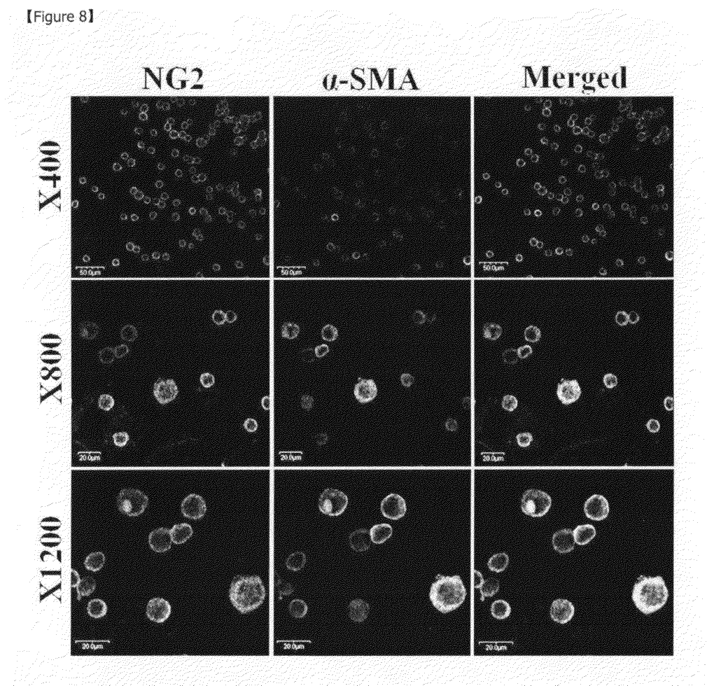

FIG. 9 shows the results of observation for the ability of the inventive human umbilical cord-derived mesenchymal stem cells to differentiate into adipocytes. After the cells were allowed to differentiate for 21 days, the cells were stained with Oil red O in order to observe the production of fatty vacuoles.

Hereinafter, the present invention will be described in further detail with reference to examples, but the scope of the present invention is not limited only to these examples.

### MODE FOR INVENTION

### I. Isolation/Identification and Freezing and Thawing of Endothelial Stem/Progenitor Cells

### Example 1

### Isolation and Culture of Endothelial Stem/Progenitor Cells from Human Umbilical Cord

1-1: Process of Obtaining Umbilical Cord

The outer surface of umbilical cord having a length of about 10-20 cm was sprayed with 75% ethanol and washed several times with sterile gauze. Then, the umbilical cord was completely immersed in HBSS (Hanks' Balanced Salt Solution, JBI, 003-02) solution and transported at room temperature. Herein, a high concentration of antibiotics (3× antibiotics, Gibco, 5240-062) were added to the solution so as to prevent the contamination of the umbilical cord during carriage/storage.

1-2: Isolation and Culture of Cells from Umbilical Cord

Umbilical cord was cut to a size of about 3-4 cm, and then incised longitudinally along the vein to expose the vascular endothelium. 0.05% collagenase I (Gibco, 17100-017) solution, pre-stored at 37° C., was poured onto a Petri dish to a height of about 0.5 cm, and the vascular endothelium was put thereon such that it faced downward.

Then, the Petri dish was stored in an incubator at 37° C. for 20 minutes, and the vascular endothelium was carefully scratched with a scraper. The detached endothelial cells were stored in a water bath at 37° C. for 30 minutes with shaking. The cells were lightly shaken at a 5-minute interval.

The cells were passed through a 70-100 μm mesh, and then centrifuged at 4° C. at 500×g for 10 minutes. After the supernatant was removed, the cells were re-suspended in RPMI 1640 (JBI, L011-01) and centrifuged at 4° C. at 500×g for 10 minutes. The suspension and centrifugation process was repeated two times, thus washing the cells.

The washed cells were suspended in 1 Ml of a culture medium (see Table 1), and then seeded in a 35-mm culture dish.

TABLE 1
Composition of medium (100 ml) for culture of
endothelial stem/progenitor cells

| Components | Volume |
|---|---|
| RPMI 1640 | 80 Ml |
| FBS | 20 Ml |
| Insulin (5 μg/Ml) | 100 μl |
| Hydrocortisone (2.4 μg/Ml) | 10 μl |
| Heparin (10 U/Ml) | 100 μl |
| Vascular endothelial cell growth factor (15 μg/Ml) | 100 μl |

1-3: Subculture

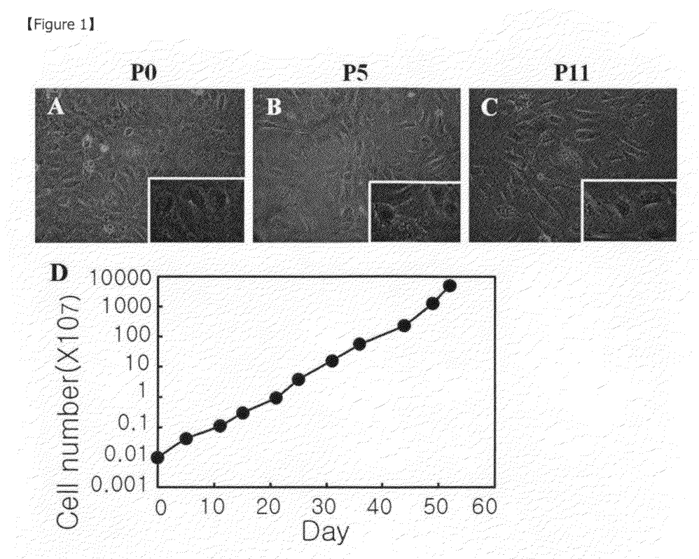

When the cells reached a confluency of 70% after the initiation of the cell isolation/culture, the cells were subcultured. This is because the cells show the highest viability and proliferation at a confluency of about 70%. FIG. 1 shows photographs of cells at 4 days of culture. In FIG. 1, (A) is a 40× photograph; (B) is a 200× photograph, and (C) is a 200× photograph, and (D) is a 400× photograph.

Specifically, the inventive method for subculturing endothelial stem/progenitor cells was carried out in the following manner. After the medium was removed from the cells, cultured in the culture dish in Example 1-2 above, the cells were washed once with RPMI 1640 to completely remove the remaining FBS. Then, 0.05% trypsin/EDTA, warmed in a water bath at 37° C. for about 10 minutes, was added to the culture dish such that the cells were dipped therein. Then, the culture dish was incubated in a CO₂ incubator for 1 minute. A light impact was applied to the culture dish to physically detach the cells. Trypsin/EDTA and the same amount of medium were added to the cells to inactivate the cells, and then the cells were collected. The collected cells were centrifuged at 4° C. at 500×g for 5 minutes to remove the supernatant. The remaining cells were suspended in a culture medium, and then the number and viability of the cells were examined using tryphan blue.

The preferred size of a culture dish for use in the cell subculture, and the preferred number of cells which are inoculated in the cell subculture according to the present invention, are shown in Table 2 below.

TABLE 2
Size of culture dish and number of cells

| Size of culture dish | Number of cells inoculated |
|---|---|
| 35 mm | 1-2 × 10⁵ |
| 60 mm | 2-4 × 10⁵ |
| 100 mm | 4-6 × 10⁵ |

### Example 2

### Measurement of Purity of Endothelial Stem/Progenitor Cells Isolated from Human Umbilical Cord

In order to measure the purity of the endothelial stem/progenitor cells, isolated and cultured according to the method of Example 1, FACS analysis and immunohistochemical/immunofluorescent analysis were performed using specific markers known to be expressed only in vascular endothelial cells. Also, in order to examine whether the isolated and cultured cells function as vascular endothelial cells, whether the isolated and cultured cells would absorb LDL (low density lipoprotein) was examined.

2-1: FACS (Fluorescence Activated Cell Sorting) Analysis

The FACS analysis of the endothelial stem/progenitor cells, isolated in Example 1, was performed using the following markers (Table 3).

TABLE 3
Markers used in FACS analysis

| Markers | Other names | Expression |
|---|---|---|
| CD34 | Sialomucin | Expressed in hematopoietic stem cells |
| CD31 | PECAM-1 | Expressed in endothelial progenitor cells |
| | | and endothelial cells |

Specifically, the FACS analysis of the endothelial stem/progenitor cells was performed in the following manner. Less than 5×10⁵ cells were placed in an FACS tube, and 2 Me of FACS buffer was added then added thereto. Then, the cell-containing medium was centrifuged at 4° C. at 500×g for 5 minutes. Only the umbilical cord-derived stem cells were suspended in 100 μl of the supernatant, and 1 μl of an FcR reagent was then added thereto. The cell suspension was cultured on ice for 30 minutes. 2 Ml of FACS buffer was added to the cells, and the cell-containing medium was centrifuged at 4° C. at 500×g for 5 minutes. The cells were treated with fluorescence-labeled antibodies, that is, CD31-FITC and CD34-PE-Cy5 or CD34-APC, and were cultured on ice for 30 minutes. Then, 2 Ml of FACS buffer was added to the cells, and the cell-containing medium was centrifuged at 4° C. at 500×g for 5 minutes. The cells were suspended in 100 μl of the supernatant, and then analyzed with an FACS calibur.

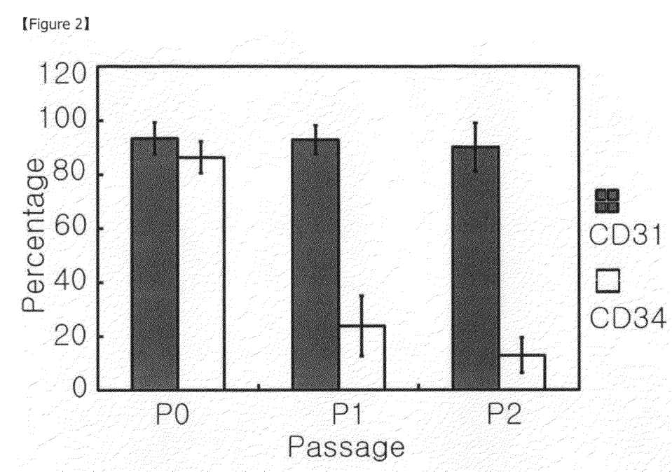

From the test results, it can be seen that, in the case of the endothelial stem/progenitor cells of the present invention, more than 90% of the cells expressed CD31, which is the marker of endothelial cells, and the expression of CD34 was also maintained, but was rapidly reduced with the progression of passages (FIG. 2 and Table 4).

TABLE 4
FACS analysis results

| Passage | CD34 | CD31 |
|---|---|---|
| 0 | 86.39 ± 5.83% | 93.42 ± 5.90% |
| 1 | 23.81 ± 11.26% | 92.95 ± 5.32% |
| 2 | 12.86 ± 6.61% | 90.19 ± 9.01% |

2-2: Immunohistochemical/Immunofluorescent Staining Analysis

The immunohistochemical and immunofluorescent analysis of the endothelial stem/progenitor cells, isolated and cultured in Example 1, was performed using the following marker (Table 5).

TABLE 5
Markers used in immunohistochemical and immunofluorescent analysis

| Markers | Other names | Expression |
|---|---|---|
| CD31 | PECAM-1 | Expressed in endothelial cells |
| vWF | von Willebrand Factor | Expressed in endothelial cells |
| α-SMA | α-smooth muscle actin | Expressed in vascular muscle cells |
| Desmin | — | Expressed in various muscle cells |

Specifically, the immunohistochemical analysis of the endothelial stem/progenitor was performed in the following manner. The endothelial stem/progenitor cells were cultured in 50 Ml of methanol containing 700 μl of H₂O₂, at room temperature for 30 minutes. Then, the cells were washed three times with PBS on a shaker for 5 minutes. The washed cells were cultured in 0.5% Triton-X 100 for 15 minutes. The cultured cells were washed three times with PBS on a shaker for 5 minutes. After removing moisture, the cells were treated with 30 μl of normal goat serum at room temperature for 1 hour. After the normal goat serum was removed, the cells were treated with 30 μl of a primary antibody and cultured at room temperature for 1 hour. Then, the cells were washed three times with PBS on a shaker for 5 minutes, and after moisture was removed, the cells were treated with 30 μl of a secondary antibody for 1 hour. Then, the cells were washed three times with PBS on a shaker for 5 minutes and were color-developed using DAB for 30-90 seconds. Then, the cells were washed with running tap water for 10 minutes. After the cell nuclei were stained with hematoxylin, the cells were washed with running tap water for 10 minutes. After the cells were treated sequentially with 70%, 85%, 95% and 100% EtOH, the cells were cultured in xylene for 10 minutes. Then, the cells were mounted with a mounting solution and covered with a cover glass. Then, the cells were observed with an optical microscope.

The immunofluorescent staining analysis of the endothelial stem/progenitor cells was performed in the same manner as in the immunohistochemical analysis, except for the following. The cells were treated with 30 μl of a fluorescence-labeled secondary antibody for 1 hour and washed three times with PBS on a shaker for 5 minutes. Then, the cells were treated with 30 μl of DAPI (4′-6-Diamidino-2-phenylindole) for 5 minutes. The treated cells were washed three times with PBS on a shaker for 5 minutes. The washed cells were mounted with a fluorescent mounting solution and covered with a cover glass. Then, the cells were observed with a confocal microscope or an optical microscope.

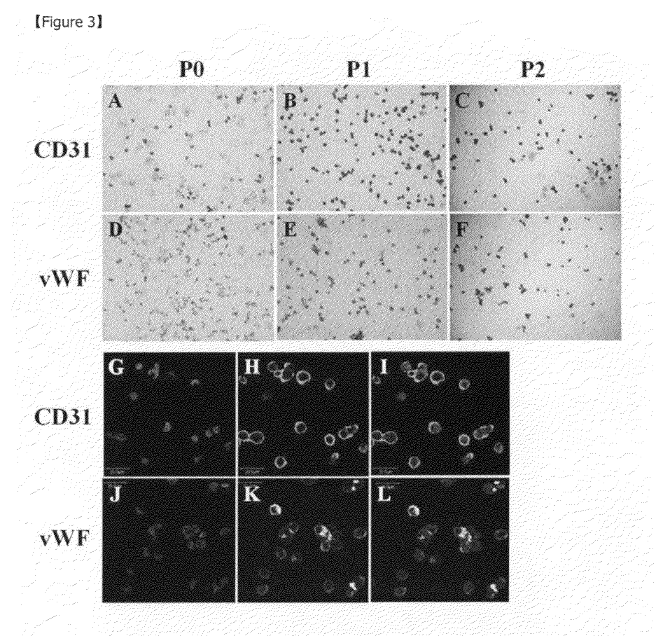

The test results showed that Desmin or α-SMA was not substantially expressed. This suggests that the endothelial stem/progenitor cells, isolated in the present invention, are not contaminated with fibroblasts. Also, in the endothelial stem/progenitor cells, isolated in the present invention, the predominant expression of markers CD31 and vWF could be observed (FIG. 3).

2-3: Absorption of LDL (Low Density Lipoprotein)

In order to examine whether the endothelial stem/progenitor cells, isolated in the present invention, show the function of vascular endothelial cells, the absorption of LDL (low density lipoprotein) was analyzed. For this purpose, 1×10⁵ endothelial stem/progenitor cells, isolated in Example 1, were cultured in a 35-mm culture dish for 2 days. Then, the supernatant was removed, and the cells were washed once with RPMI 1640. After Dil-labeled ac LDL (1,1-dioctadecyl-3,3,3′,3′-tetramethylindo carbocyanine perchlorate-labeled ac-LDL) was added to a medium to a concentration of 10 μg/Ml, the cells were cultured in the medium for 4 hours. The supernatant was removed, and the cells were washed once with RPMI 1640. The washed cells were observed with a fluorescent microscope or analyzed by FACS.

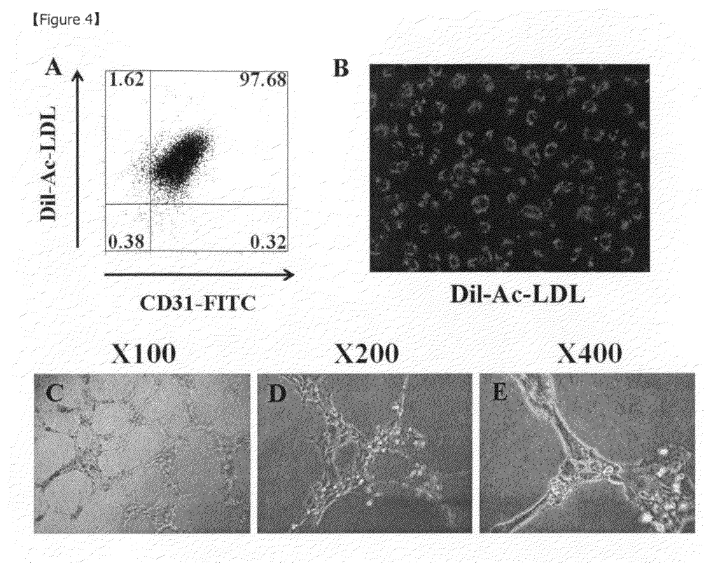

The results of FACS analysis of LDL absorption showed that the cells expressing CD31 absorbed LDL (FIG. 4A). The results of fluorescent microscopic observation of LDL absorption showed that LDL was located in the cytoplasm (FIG. 4B).

### Example 3

### Freezing and Thawing of Endothelial Stem/Progenitor Cells

The endothelial stem/progenitor cells, isolated and identified according to the present invention, was frozen and stored in the following manner.

1-2×10⁶ endothelial stem/progenitor cells, isolated and cultured in Example 1, were carefully suspended in 1 Ml of a freezing medium, and then placed in a freezing vial, which was then placed in a cryogenic box, stored at room temperature. Then, the cell-containing freezing vial in the cryogenic box was stored at −80° C. for one day. Then, the freezing vial was transferred and stored in a liquid nitrogen (LN2) tank. The preferred composition of the freezing medium (50 ml) for use in the present invention is shown in Table 6 below.

TABLE 6
Composition of freezing medium

| Components | Contents |
|---|---|
| DMSO | 5 Ml |
| FBS (or HBS) | 10 Ml |
| RPMI1640 | 35 Ml |

Also, the inventive endothelial stem/progenitor cells, stored in the above manner, were thawed and used when needed. The above-frozen freezing vial was rapidly thawed at 37° C. and was transferred to a clean bench before it was completely thawed. Then, the cells were added dropwise to 9 ml of medium using a pipette. The cell-containing medium was centrifuged at 4° C. at 500×g for 5 minutes, and the cells were collected, suspended in 1 Ml of medium and seeded in a culture dish.

### II. Isolation/Identification and Freezing/Thawing of Human Umbilical Cord-Derived Mesenchymal Stem Cells

### Example 4

### Isolation and Culture of Cells from Umbilical Cord

4-1: Process of Obtaining Umbilical Cord

Umbilical cord having a length of about 10-20 cm was completely dipped in an HBSS (Hanks Balanced Salt Solution, JBI, 003-02) solution containing 3× antibiotics (Gibco, 5240-062), gentamycin and plasmocin (Invivogen, ant-mpt), and were transported at room temperature.

4-2: Isolation and Culture of Cells from Umbilical Cord

Umbilical cord was cut to a size of about 3-4 cm, and then incised along the outer portion of the artery using surgical scissors. Then, the artery was separated from the umbilical cord using tweezers. 0.05% solution I [0.05% collagenase type I (Gibco, 17100-017), 0.02% pronase (Roche, 165 921), and 0.2% DNase I (Sigma)], pre-stored at 37° C., was poured into a 50-Ml tube to a level of about 5-10 Ml, and the separated artery was dipped in the solution in the tube.

Then, the tube was incubated in a incubator at 37° C. for 2, 4 and 6 hours, and the supernatant was collected and passed through a 30-μm mesh. After the remaining tissue was filled with HBSS up to a volume of 50 Ml, it was centrifuged was conducted at 4° C. at 500×g for 10 minutes. After the supernatant was removed, the tissue was re-suspended in 50 Ml of DMEM (JBI, L001-05), and then centrifuged at 4° C. at 500×g for 10 minutes. The suspension and centrifugation process was repeated twice.

Finally, after the supernatant was removed, the cells were suspended in 1 Ml of culture medium (80 Ml of DMEM (JBI, L001-05), 20 Ml of FBS (Hyclone, SH30088.03) (Table 7), and then seeded in a 35-mm culture dish.

TABLE 7
Composition of medium for culture of
mesenchymal stem cells (100 Ml)

| Components | Volume |
|---|---|
| DMEM | 80 Ml |
| FBS | 20 Ml |

4-3: Subculture

When a confluency of 70% is reached after the initial isolation/culture of human umbilical cord-derived mesenchymal stem cells, the cells were subcultured. This is because the cells show the highest viability and proliferation at a confluency of about 70%.

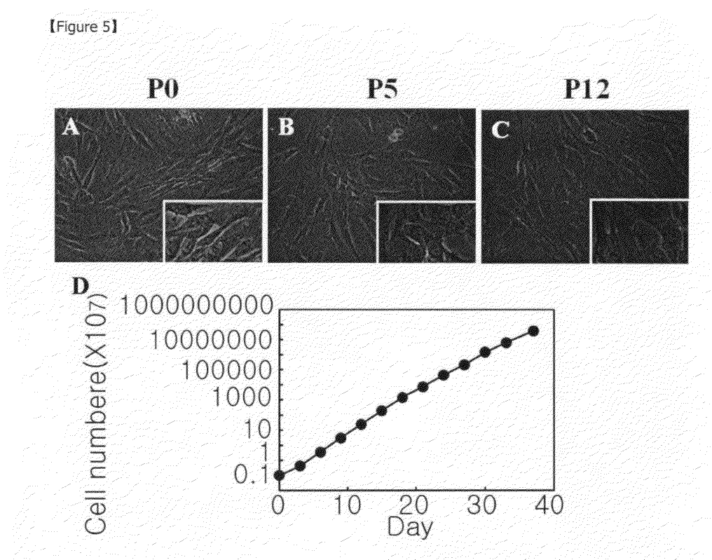

Photographs of the human umbilical cord-derived mesenchymal stem cells at passages 0-12 are shown in FIG. 5. As shown in FIG. 5, the cells maintained a spindle shape similar to that of fibroblasts at early passage to late passage. It could be confirmed that this shape was similar to that of human bone marrow-derived mesenchymal stem cells.

Specifically, the method for subculturing human umbilical cord-derived mesenchymal stem cells according to the present invention was performed in the following manner. After the medium was removed from the cells at a confluency of 70%, the cells were washed once with DMEM to completely remove the remaining FBS. 0.05% trypsin/EDTA, warmed in a water bath at 37° C. for about 10 minutes, was added to the cells such that the cells were dipped therein, the cells were incubated in a CO₂ incubator for 1 minute. A light impact was applied to the culture dish to physically detach the cells. Trypsin/EDTA and the same amount of medium were added to inactivate the cells, and then the cells were collected. The cells were centrifuged at 4° C. at 500×g for 5 minutes to remove the supernatant, the remaining cells were suspended in medium, and the number and viability of the cells were examined using tryphan blue. Herein, the culture medium shown in Table 7 was used.

Also, the number of cells, seeded in the cell subculture process according to the present invention, and the size of a culture dish for use in the cell subculture, preferably satisfy the values shown in Table 8 below.

TABLE 8
Size of culture dish and number of cells seeded

| Size of culture dish | Number of cells seeded |
|---|---|
| 35 mm | 0.5 × 10⁵ |
| 60 mm | 1 × 10⁵ |
| 100 mm | 3 × 10⁵ |

A growth curve of the human umbilical cord-mesenchymal stem cells during subculture is shown in FIG. 5. As can be seen in FIG. 5, during the subculture period, the cells showed no senescence phenomenon and stably proliferated (FIG. 5).

### Example 5

### Measurement of Purity of Mesenchymal Stem Cells Isolated from Human Umbilical Cord

In order to measure the purity of the mesenchymal stem cells, isolated and cultured according to the method of Example 4, FACS analysis and immunohistochemical analysis were performed using markers known to be expressed only in vascular endothelial cells.

5-1: FACS (Fluorescence Activated Cell Sorting) Analysis

For the FACS analysis of the human umbilical cord-derived mesenchymal stem cells, isolated and cultured in the present invention, the markers shown in Table 9 below were used.

TABLE 9
Markers used in FACS analysis

| Markers | Other names | Expression |
|---|---|---|
| CD29 | β1 integrin | Lymphocytes, smooth muscle cells, |
| | | and epithelial cells |
| CD31 | PECAM-1 | Vascular endothelial cells |
| CD44 | H-CAM | Lymphocytes and mesenchymal stem |
| | | cells |
| CD45 | Leukocyte common | Hematopoietic cells |
| | antigen | |
| CD73 | Ecto-5′ nucleotidase | Mesenchymal stem cells |
| CD90 | Thy-1 | Hematopoietic stem cells and |
| | | mesenchymal stem cells |
| CD105 | Endoglin | Vascular endothelial cells and |
| | | mesenchymal stem cells |
| CD117 | c-kit | Hematopoietic stem cells |
| HLA-DR | | |
| HLA-I | | |

More specifically, the FACS analysis was performed in the following manner. Less than 5×10⁵ mesenchymal stem cells, isolated and cultured in Example 4 above, were placed in a FACS tube, and then 2 Ml of FACS buffer was added thereto, and the cell-containing medium was centrifuged at 4° C. at 500×g for 5 minutes, and the supernatant was removed. In order to analyze an antigen in the cells, the cells were suspended in 500 μl of PBS, and then 1200 μl of ETOH, stored on ice, was added dropwise thereto with vortexing. Then, the cells were cultured on ice for 30 minutes, 2 Ml of FACS buffer was added thereto, and the cell-containing medium was centrifuged at 4 at 500×g for 5 minutes. The cells were suspended in 100 μl of the supernatant, and the cell suspension was treated with a fluorescence-labeled antibody and cultured on ice for 30 minutes. 2 Ml of FACS buffer was added to the cells, and the cell-containing medium was centrifuged at 4° C. at 500×g for 5 minutes. Then, the cells were suspended in 100 μl of the supernatant and analyzed with FACSCalubur.

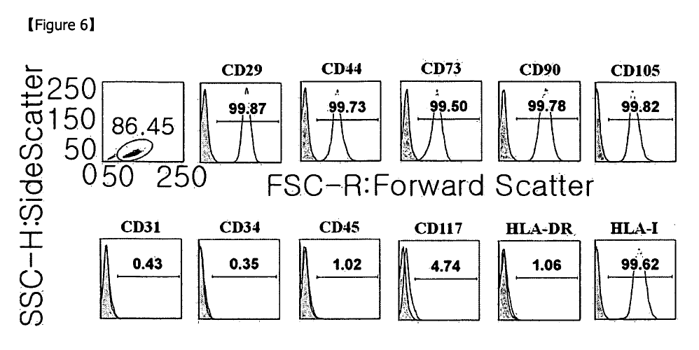

The test results showed that CD29, CD44, CD73, CD90, CD105 and HLA-I were expressed in the human umbilical cord-derived mesenchymal stem cells at expression levels of more than 99% (FIG. 6). This suggests that the mesenchymal stem cells, isolated and cultured in the present invention, were very uniform and highly pure.

5-2: Immunohistochemistry

For the immunohistochemical analysis of the human umbilical cord-derived mesenchymal stem cells, isolated and cultured in the present invention, the markers shown in Table 10 below were used.

TABLE 10
Markers used in immunohistochemical analysis

| Markers | Other names | Expression |
|---|---|---|
| NG2 | NG2 Chondroitin Sulfate | Oligodendrocytes |
| | Proteoglycan | |
| a-SMA | a-smooth muscle actin | Various muscle cells, mesenchymal |
| | | cells and mesenchymal stem cells |
| Desmin | — | Expressed in various muscle cells |

Specifically, the immunohistochemical analysis was performed in the following manner. First, the mesenchymal stem cells, isolated and cultured in Example 4, were cultured in 50 ml of methanol, containing 700 μl of H₂O₂, at room temperature for 30 minutes. Then, the cells were washed three times with PBS on a shaker for 5 minutes. Then, the cells were cultured in 0.5% Triton-X100 for 15 minutes and washed three times with PBS on a shaker for 5 minutes. After moisture was removed, the cells were treated with 30 μl of normal goat serum at room temperature for 1 hour. After the normal goat serum was removed, the cells were treated with 30 μl of a primary antibody and cultured at room temperature for 1 hour. Then, the cells were washed three times with PBS on a shaker for 5 minutes, and after moisture was removed, the cells were treated with 30 μl of a secondary antibody for 1 hour. Then, the cells were washed three times with PBS on a shaker for 5 minutes. The cells were color-developed using DAB for 30-90 seconds, and then washed with running tap water for 10 minutes. The cell nuclei were stained with hematoxylin, and then the cells were washed with running tap water for 10 minutes. After the cells were treated sequentially with 70%, 85%, 95% and 100% EtOH, the cells were cultured in xylene for 10 minutes. Then, the cells were mounted with a mounting solution and covered with a cover glass. Then, the cells were observed with an optical microscope.

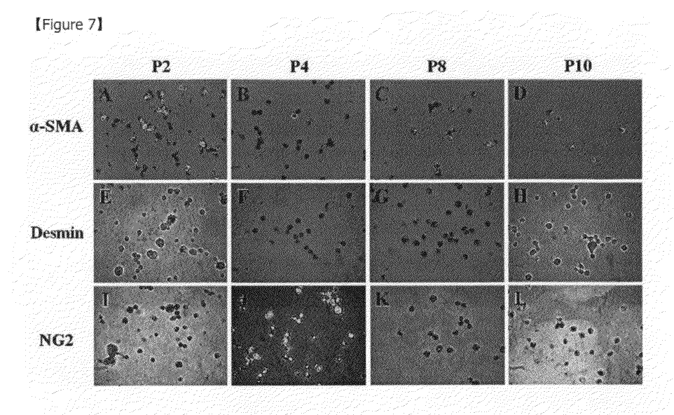

From the test results, it can be seen that NG2 and α-SMA were expressed in most of the cells at early passage to late passage (FIG. 7). Also, it could be observed through immunofluorescent staining that NG2 and α-SMA were simultaneously expressed in most of the cells.

### Example 6

### Differentiation of Mesenchymal Stem Cells

The differentiation of the human umbilical cord-derived mesenchymal stem cells, isolated and cultured in the present invention, into adipocytes, was induced. Specifically, after the cells were grown to fill up the dish, the medium was replaced with a medium for inducing differentiation into adipocytes, and the cells were cultured for 21 days. The culture medium was replaced with a fresh medium at 3-day intervals, and fat in the cells was observed by staining with Oil red O. The composition of the medium for inducing differentiation into adipocytes is shown in Table 11.

TABLE 11
Composition of medium for inducing differentiation into adipocytes

| Components | Concentration | |
|---|---|---|
| Indomethacin | 50 | μM |
| 3-isobutyl-1-methylxanthine (IBMX) | 0.5 | mM |
| Dexamethasone | 1 | μM |
| Insulin | 10 | μg/Ml |
| DMEM | 95 | Ml |
| FBS | 5 | Ml |

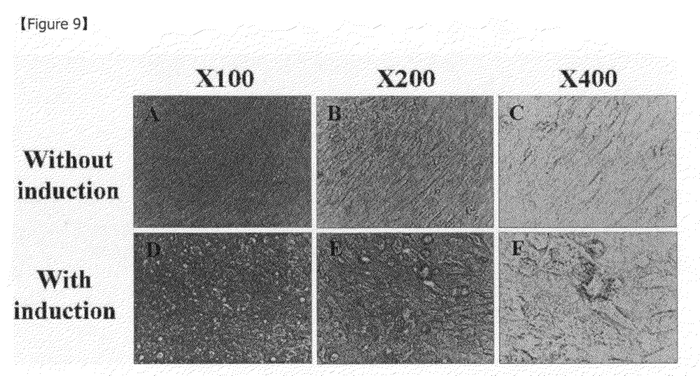

The test results showed that the mesenchymal stem cells could differentiate into adipocytes. Also, the results of Oil red O staining showed that lipid vacuoles in the cytoplasm were stained with red (FIG. 9).

### Example 7

### Freezing and Thawing of Mesenchymal Stem Cells

The human umbilical cord-derived mesenchymal stem cells, isolated and identified according to the present invention, were frozen and stored in the following manner. First, 1-2×10⁶ cells were prepared, carefully suspended in 1 Ml of a freezing medium and placed in a freezing vial, which was then placed in a cryogenic box, stored at room temperature. Then, the freezing vial in the cryogenic box was stored at −80° C. for one day. Then, the freezing vial was transferred and stored in a liquid nitrogen (LN2) tank. The preferred composition of the freezing medium (100 Ml) used in the present invention is shown in Table 12 below.

TABLE 12
Composition of freezing medium (100 Ml)

| Components | Contents |
|---|---|
| DMSO | 5 Ml |
| FBS | 10 Ml |
| DMEM | 35 Ml |

Also, the human umbilical cord-derived mesenchymal stem cells, stored according to the above method, were thawed in the following manner and used when needed. The freezing vial was rapidly thawed at 37° C. and transferred to a clean bench before it was completely thawed. The cells were added dropwise to 9 Ml of medium using a pipette. The cell-containing medium was centrifuged at 4° C. at 500×g for 5 minutes, and the centrifuged cells were suspended in 1 Ml of medium and seeded in a dish. The preferred composition of the thawing medium used in the present invention is shown in Table 13.

TABLE 13
Composition of thawing medium (100 Ml)

| Components | Contents |
|---|---|
| FBS | 20 Ml |
| DMEM | 80 Ml |

### INDUSTRIAL APPLICABILITY

As described above, according to the method of the present invention, endothelial stem/progenitor cells and mesenchymal stem cells can be easily isolated and purified with high purity from umbilical cord, and can be cultured with high viability for a long period of time.

1. A method for isolating and culturing endothelial stem/progenitor cells and mesenchymal stem cells from umbilical cord, the method comprising the steps of:
(a) culturing any one selected from the umbilical cord-derived vascular endothelium, blood vessel and blood vessel-removed umbilical cord of mammals, including human beings, together with protease or protease and DNA-degrading enzyme;
(b) scratching the cultured endothelium of step (a) with a scraper to collect a cell mass, and collecting a supernatant of the cultured blood vessel and blood vessel-removed umbilical core of step (a);
(c) isolating and purifying endothelial stem/progenitor cells from the cell mass of step (b), and isolating and purifying mesenchymal stem cells from the supernatant of step (b); and
(d) culturing the endothelial stem/progenitor cells or mesenchymal stem cells, isolated and purified in the step (c).

2. The method of claim 1, wherein the mammals are human beings.

3. The method of claim 1, wherein the protease is collagenase and/or pronase.

4. The method of claim 1, wherein the DNA-degrading enzyme is DNase.

5. The method of claim 1, wherein the vascular endothelium in the step (a) is obtained by longitudinally incising the vein of the umbilical cord.

6. The method of claim 1, wherein the blood vessel in the step (a) is an artery separated by cutting along the Wharton's jelly of the umbilical cord.

7. The method of claim 1, wherein the culture of the vascular endothelium in the step (a) is carried out at 35-38° C. for 15-25 minutes, after the vascular endothelium is treated with collagenase.

8. The method of claim 1, wherein the culture of the blood vessel and the blood vessel-removed umbilical cord in the step (a) is carried out at 35-38° C. for 2-6 hours, after the blood vessel or the blood vessel-removed umbilical cord is treated with collagenase, pronase and DNase.

9. The method of claim 1, wherein the isolation and purification of the endothelial stem/progenitor cells in the step (c) are carried out by culturing the cell mass of step (b) in a water bath, and then filtering and washing the cultured cells.

10. The method of claim 9, wherein the culture is carried out with shaking at 35-38° C. for 20-40 minutes.

11. The method of claim 9, wherein the filtration of the cultured cells is carried out by passing the cells through a mesh having a pore size of 0.1-0.7 μm.

12. The method of claim 9, wherein the washing of the cultured cells is carried out by treating the cultured cells with a medium, and then centrifuging the cell-containing medium to remove a supernatant.

13. The method of claim 1, wherein the culture of the endothelial stem/progenitor cells in the step (d) is carried out by suspending the endothelial stem/progenitor cells, isolated and purified in the step (c), in a culture medium, and seeding the suspended cells in a culture dish.

14. The method of claim 13, wherein the culture medium contains RPMI 1640, FBS, insulin, hydrocortisone, heparin and an endothelial cell growth factor.

15. The method of claim 1, wherein the isolation and purification of the mesenchymal stem cells in the step (c) is carried out by filtering and washing the supernatant obtained in the step (b).

16. The method of claim 14, wherein the filtration is carried by passing the supernatant through a mesh having a pore size of 10-100 μm.

17. The method of claim 1, wherein the culture of the mesenchymal stem cells in the step (d) is carried out by suspending the mesenchymal stem cells, isolated and purified in the step (c), in a culture medium, and seeding the suspended cells in a culture dish.

18. The method of claim 17, wherein the culture medium contains DMEM and FBS at a volume ratio of 4:1.

19. The method of claim 1, which additionally comprises a step of subculturing the cells, when the cells reach a confluency of 60-80% after the initiation of the culture in the step (d).

20. The method of claim 19, wherein the subculture comprises the steps of:
(i) washing the isolated and purified endothelial stem/progenitor cells or mesenchymal stem cells;
(ii) placing the endothelial stem/progenitor or mesenchymal stem cells of step (i) in a culture dish, adding trypsin/EDTA to the cells, and then incubating the cells in a CO₂ incubator;
(iii) applying a light impact to the culture dish of step (ii) to physically detach the cells, adding typsine/EDTA and the same amount of culture medium to the cells to inactivate the cells, and then collecting the cells; and
(iv) centrifuging the cells at low temperature, removing the supernatant, re-suspending the centrifuged cells in culture medium and transferring the cell suspension to another culture dish.

21. The method of claim 20, wherein the washing in the step (i) is carried out using RPMI 1640 or DMEM.

22. The method of claim 20, wherein the trypsin/EDTA in the step (ii) is warmed in a water bath at 35-38° C. for 5-15 minutes.

23. The method of claim 20, wherein the incubation in the step (ii) is carried out for 0.5-1.5 minutes.

24. The method of claim 20, which additionally comprises, after the centrifugation in the step (iv), a step of staining the cells from which the supernatant was removed, and monitoring the number or viability of the cells.

25. Umbilical cord-derived endothelial stem/progenitor cells obtained according to the method of claim 1.

26. The umbilical cord-derived endothelial stem/progenitor cells of claim 25, which do not express desmin and α-SMA (α-smooth muscle actin), but highly express CD31, CD34 and vWF (von Willebrand Factor).

27. The umbilical cord-derived endothelial stem/progenitor cells of claim 25, which have an ability to absorb LDL (low density lipoprotein).

28. Umbilical cord-derived mesenchymal stem cells obtained according to the method of claim 1.

29. The mesenchymal stem cells of claim 28, which highly express CD29, CD44, CD73, CD90, CD105, α-SMA (α-smooth muscle actin) and NG2 (NG2 Chondroitin Sulfate Proteoglycan).

30. A method for freezing and storing umbilical cord-derived endothelial stem/progenitor cells and mesenchymal stem cells, the method comprising the steps of:
(a) suspending the cells of claim 25 or 28 in a freezing medium, placing the cell suspension in a freezing vial, and placing the cell-containing freezing vial in a cryogenic box, stored at room temperature; and
(b) storing the cell-containing freezing vial in the cryogenic box at −75 to 85° C. for 20-30 hours, and then transferring and storing the cell-containing freezing vial in a liquid nitrogen (LN2) tank.

31. The method of claim 30, wherein the freezing medium in the step (a) contains DMSO, FBS and RPMI1640 at a ratio of 5:10:35.

32. A method for thawing umbilical cord-derived endothelial stem/progenitor cells and mesenchymal stem cells, the method comprising the steps of:
(a) rapidly thawing a freezing vial, containing cells frozen according to the method of claim 30, at a temperature of 35-38° C., and transferring the freezing vial to a clean bench before it is completely thawed;
(b) adding the cells, obtained in the step (a), drop-by-drop to a thawing medium using a pipette, and then centrifuging the cell-containing medium at a temperature of 2-5° C.; and
(c) suspending the centrifuged cells in a culture medium and seeding the suspended cells in a culture dish.

33. The method of claim 32, wherein the thawing medium in the step (b) contains FBS and DMEM at a volume ratio of 2:8.
